(19) United States
(12) Patent Application Publication
(10) Pub. No.: US 2026/0035232 A1
(43) Pub. Date: Feb. 5, 2026

(54) PAIR OF GOGGLES FOR ANIMALS OF THE EQUINE SPECIES

(71) Applicant: EQUIDIVA, Saint-Léger en Yvelines (FR)

(72) Inventor: Pascale Latour, Saint-Leger-en-Yvelines (FR)

(21) Appl. No.: 19/099,977

(22) PCT Filed: Aug. 3, 2023
(86) PCT No.: PCT/EP2023/071511

§ 371 Date: Jan. 30, 2025

(30) Foreign Application Priority Data
Aug. 4, 2022 (FR) FR2208091

(51) Int. Cl. B68C 5/00; A61F 9/02

(52) U.S. Cl. CPC B68C 5/00; A61F 9/026; A61F 2009/021

(57) ABSTRACT

The invention relates to a pair of goggles (1) intended to be fitted to the head (100) of an animal of the equine species, the pair of goggles (1) comprising a set of straps (20) and two shields (11, 12). Each shield (11, 12) comprises a sleeve (3) that extends between a first sleeve end (31) and a second sleeve end (32). The first sleeve end (31) is provided with a first hole (41) and the second sleeve end (32) is provided with a second hole (42). The second sleeve end (32) is provided with a collar (5) fitted to a rim (4) of the second hole (42).

### TECHNICAL FIELD

The invention concerns a pair of goggles for equines, such as horses, donkeys, ponies or the like. It concerns a pair of goggles to be fitted on an equine.

### PRIOR ART

US 2010/0083914 describes a pair of goggles to be fitted on an equine, particularly a horse. The goggles are designed to protect the horse's eyes from impact. The pair of goggles comprises two shields each designed to cover one of the horse's eyes. The shields are held in place on the horse's head by a set of straps which connect the shields together and hold them in place. Each shield can be fitted with an insect screen or lens.

### TECHNICAL PROBLEM

In view of the foregoing, one problem which the present invention sets out to solve is to improve such a pair of goggles to provide better visual comfort for an equine, such as a horse, donkey, pony or the like, hereinafter referred to as a horse. Horses with visual impairments are prone to clumsiness, which can injure them and those around them. In some cases, this can lead to the horse being euthanized, which should be avoided at all costs.

It is therefore desirable to improve the shape of each of the shields to better adapt it to the physiognomy of the horse's head. It is also desirable to improve the shape of each of the shields, so that an arrangement of a lens carried by a shield is improved in relation to the eye of the horse to which the shield is fitted.

It is also desirable to improve ease of wear for the horse, which is naturally reluctant to wear such a pair of goggles.

It is even more desirable for such a pair of goggles to be able to protect sensitive eyes or eyes suffering from eye pathologies, such a pair of goggles being arranged to protect the horse's eyes from natural elements, wind, dust, or ultra-violet rays.

It is also desirable for such goggles to be adapted to enable a horse to perform tasks common to all horses, such as dressage, mounting, work in the agricultural, mainly forestry sectors, fishing, working with disabled people or the like, all of which are expanding rapidly. In other words, it is desirable for such a pair of goggles will improve the horse's life by enabling it to continue performing tasks that the horse is used to doing without a pair of goggles.

### TECHNICAL SOLUTION

The object of the present invention is a pair of goggles to be fitted to the head of an equine. The pair of goggles comprises a set of straps and two shields, each shield comprising a sleeve that extends between a first sleeve end and a second sleeve end. The first sleeve end is provided with a first hole and the second sleeve end is provided with a second hole.

According to the present invention, the second sleeve end is provided with a collar around the rim of the second hole.

The pair of goggles advantageously comprises any one or more of the following features, taken alone or in combination:
the collar and sleeve form a first angle of between 90° and 120°.
the collar extends between an topside and an underside which is equipped with a crown.
the collar is provided with a plurality of channels extending between the topside and underside of the collar.
the channels are radially interposed between the crown and the periphery around a general extension axis of the sleeve.
the collar comprises two tabs which are diametrically opposed to each other with respect to the general extension axis.
a first tab is arranged in a first plane and a second tab is arranged in a second plane, which form a second angle between them of between 70° and 120°.
the sleeve extends between at least one pair of distant points and one pair of near points, the pair of distant points being whichever one of the respective pairs of points of the first sleeve end and the second sleeve end for which a first distance between a first distant point and a second distant point is greatest, the second pair of near points being those respective pairs of points of the first sleeve end and the second sleeve end for which a second distance between a first close point and a second close point is smallest, a ratio of the first distance to the second distance is between 8 and 16.
the pair of distant points and the pair of near points are diametrically opposed with respect to the general extension axis.
the pair of distant points is equidistant from the first and second tabs.
the pair of near points is equidistant from the first and second tabs.
the pair of distant points is equidistant from the first and second tabs.
each shield comprises at least two longitudinal bosses which extend between the first sleeve end and an outer collar edge and which jointly border an inflection line which also extends between the first sleeve end and an outer collar edge.
the first hole is fitted with a corrective lens.

### BRIEF DESCRIPTION OF THE DRAWINGS

The invention and its advantages will be better understood on reading the following description and non-limiting embodiments, with reference to the appended figures, wherein:

FIG. 1 is a side view of a horse's head fitted with a pair of goggles of the present invention.

FIG. 2 is a front view of the horse's head shown in FIG. 1, wearing the goggles of the present invention.

FIG. 3 is a side view of a shield of the pair of goggles shown in the preceding figures.

FIG. 4 is a cross-sectional view of the shield shown in FIG. 3.

FIG. 5 is a bottom view of the shield shown in FIGS. 3 and 4.

### DETAILED DESCRIPTION

The present invention relates to a pair of goggles 1 intended to be fitted to a head 100 of an equine, the pair of goggles 1 comprising a set of straps 20 and two shields 11, 12, each shield 11, 12 comprising a sleeve 3 which extends between a first sleeve end 31 and a second sleeve end 32, the first sleeve end 31 being provided with a first hole 41 and the second sleeve end 32 being provided with a second hole 42.

Said second end of the sleeve 32 is provided with a collar 5 fitted to the rim 4 of the second hole 42.

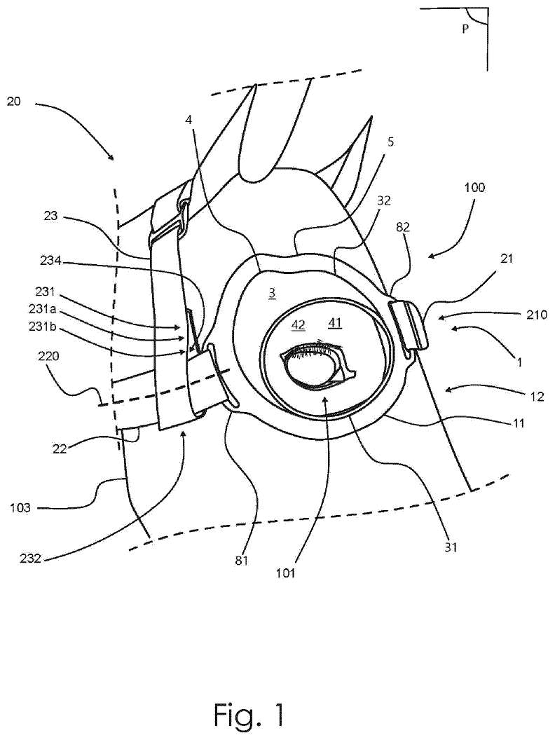

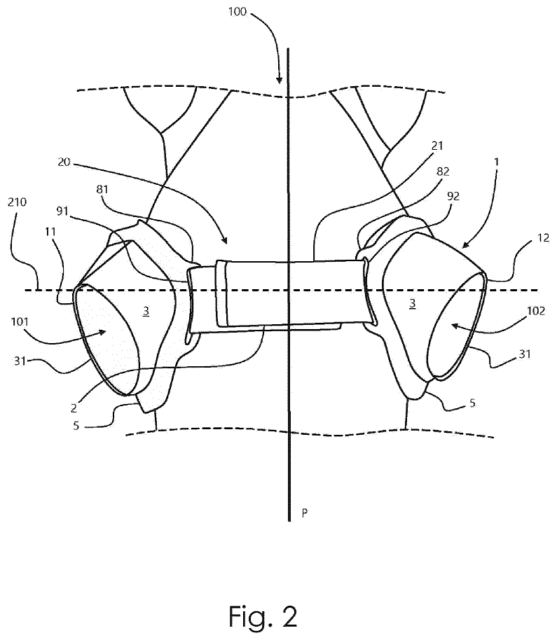

In FIGS. 1 and 2, the head 100 of an equine is fitted with a pair of goggles 1 according to the present invention. Note that such an equine may be a horse, donkey, pony or similar. The following description focuses on a pair of goggles 1 intended to be fitted to the head 100 of a horse, but can be transposed to any equine. The pair of goggles 1 comprises two shields 11, 12, including a first shield 11 intended to be fitted to the horse's right eye and a second shield 12 intended to be fitted to a left eye. The first shield 11 and the second shield 12 are usually symmetrical with respect to a plane of symmetry P interposed between them. In other words, one of the shields 11, 12 is obtained from an orthogonal symmetry with respect to the plane of symmetry P interposed between the two shields 11, 12. When the pair of goggles 1 is fitted to the horse, the plane of symmetry P is preferentially a sagittal plane of the horse. In another variant, the first shield 11 and the second shield 12 are not symmetrical with respect to the sagittal plane of the horse.

A set of straps 20 is provided to hold the shields 11, 12 on the horse's head 100 in a reliable, precise and sturdy manner without causing discomfort to the horse, and more importantly without risk of injury. More specifically, the set of straps 20 is designed to hold each shield 11, 12 around an eye 101, 102 of the respective horse to which the shield 11, 12 is fitted. The set of straps 20 comprises a front strap 21 which is intended to connect the two shields 11, 12 together on a front line 210 substantially connecting the two eyes 101, 102 of the horse. In particular, the front strap 210 is adjustable according to the width of the horse's muzzle. The set of straps 20 further comprises a lower strap 22 which is intended to also connect the two shields 11, 12 together by extending along a lower line 220 which passes beneath an underthroat 103 of the horse. The lower line 220 extends substantially in alignment with the front line 210. The set of straps 20 further comprises an upper strap 23 which extends between two ends 231 provided with buckles 232 inside which the lower strap 22 slides. It is understood that each buckle 232 delimits an internal space 234 suitable for receiving a portion of the lower strap 22. Each buckle 232, for example, is produced by folding the end 231 of the upper strap 23 onto itself, the end 231 of the upper strap 23 being provided with first hooking elements 231a cooperating with second hooking elements 231b fitted to the upper strap 23 to form said buckle 232. It is understood that the hooking elements 231a, 231b are reversible connecting elements to facilitate the fitting and removal of the upper strap 23 on the lower strap 22. It should also be noted that each of the straps 21, 22, 23 is provided with means for adjusting 2 the length of the strap 21, 22, 23 to facilitate and optimize the positioning of the pair of goggles 1 on the horse's head 100 and the stability of the pair of goggles 1 on the horse's head 100.

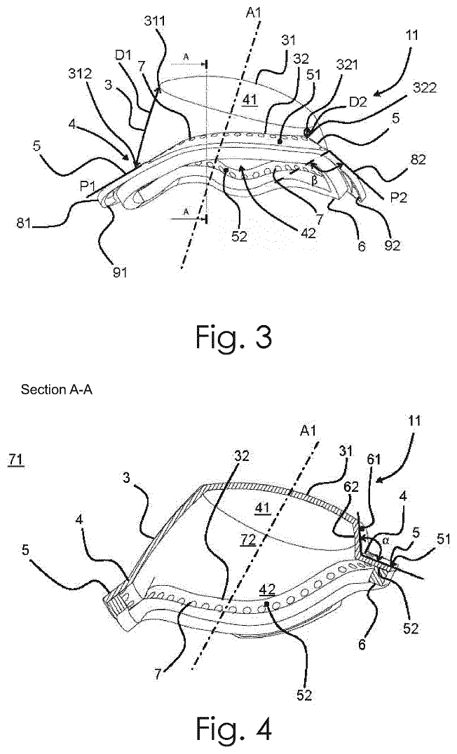

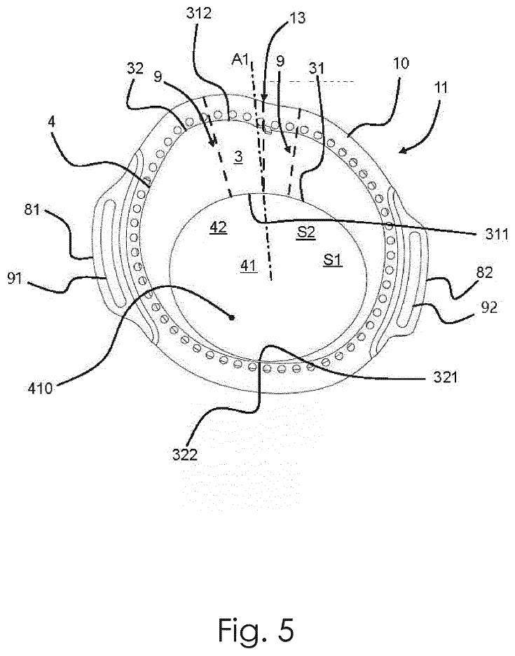

FIGS. 3 to 5 show one of the two shields 11, 12, in this case the first shield 11. At this stage of the description, it should be noted that what will be said for the first shield 11 can be transposed to the second shield 12, due to the symmetry mentioned in the above variant. Each shield 11, 12 comprises a sleeve 3 that extends axially between a first sleeve end 31 and a second sleeve end 32. It is understood that the sleeve 3 extends mainly along a general extension axis A1. The first sleeve end 31 is provided with a first hole 41 and the second sleeve end 32 is provided with a second hole 42. The first hole 41 and the second hole 42 are arranged opposite each other. The general extension axis A1 passes through each of the holes 41, 42. The general extension axis A1 is equidistant from the edges that delimit each of the holes 41, 42. Note that the first hole 41 has a first surface S1 which is smaller than a second surface S2 of the second hole. Preferentially, the first surface corresponds to 60% of the second surface S2, to within +/−10%.

The second hole 42 is delimited by a rim 4 which is provided with a collar 5. At each point on the rim 4, the collar 5 and sleeve 3 form a first angle a of between 90° and 120°, as shown in FIG. 4. In this way, the collar 5 forms a bearing surface for the shield 11, 12 on the horse's head 100. In particular, the collar 5 is shaped to match the skeleton and/or musculature of the horse's head 100, to optimize the fit of the goggles 1 on the horse's head 100. In this way, the collar 5 comprises at least one inflection point and/or circumvolution enabling the shield 11, 12 to be adapted to the horse's head 100.

The collar 5 extends axially between a topside 51, which adjoins an outer face 61 of the sleeve 3, and an underside 52, which adjoins an inner face 62 of the sleeve 3. The collar 5 has the same thickness as the shield 11, 12.

The underside 52 of the collar 5 is provided with a crown 6 which extends around the second hole 42 along the rim 4. The crown 6 is preferably made of a cellular material, such as foam or the like, to form a buffer between the shield 11, 12 and the horse's head 100. In this way, the crown 6 prevents any irritation, discomfort or injury caused by the underside 52 of the collar 5 rubbing against the horse's skin. Also, the crown 6 preferably has a parallelepiped cross-section to make it easier to attach, by gluing or similar means, onto the underside 52, and to make it easier to place against the horse's skin. The crown 6 is preferentially made of a material chosen to be soft, non-irritating, and physiologically suitable for contact with the horse's skin.

The collar 5 is provided with a plurality of channels 7 extending between the topside 51 and underside 52 of the collar 5. These channels 7 form ventilation channels to allow air to circulate between an environment external 71 to the shield 11, 12 and an interior volume 72 of the shield 11, 12. These provisions are designed to prevent condensation or fogging inside the shield 11, 12. In a preferred embodiment, these channels 7 are distributed along the rim 4 at a constant distance therefrom, the channels 7 being equally spaced around the rim 4. The channels 7 are radially interposed between the crown 6 and the periphery 4 around a general extension axis A1 of the sleeve 3.

Referring in particular to the figure, the collar 5 comprises two tabs 81, 82 which are diametrically opposed to each other with respect to the general extension axis A1. The tabs 81, 82 are radial protrusions of the collar 5. A first tab 81 lies substantially in a first plane P1 and a second tab 82 lies substantially in a second plane P2. Note that the first plane P1 and the second plane P2 intersect, and form, for example, a second angle P of between 70° and 120°.

Each tab 81, 82 is provided with a window 91, 2 for the passage of one end of one of the front 21 or bottom 22 straps. Each window 91, 92 opens out through the topside 51 and underside 52 of collar 5. It is thus understood that the first tab 81 is provided with a first window 91 and the second tab 82 is provided with a second window 92. For example, the windows have an oblong shape centered on the general extension axis A1.

It should be noted that the sleeve 3 extends between at least one pair of distant points 311, 312, including a first distant point 311 and a second distant point 312, and a pair of near points 321, 322, including a first near point 321 and a second near point 322. The first distant point 311 and the first near point 321 form the first sleeve end 31 and delimit the first hole 41. The second distant point 312 and the second near point 322 form the second sleeve end 32 and delimit the second hole 42. The pair of distant points 311, 312 is whichever one of the respective pairs of points of the first sleeve end 31 and the second sleeve end 32 for which a first distance D1 between the first remote point 311 and the second remote point 312 is greatest, such a first distance D1 being taken between these points orthogonally to the first sleeve end 31 and the second sleeve end 32. The pair of near points 321, 322 is whichever one of the respective pairs of points of the first sleeve end 31 and the second sleeve end 32 for which a second distance D2 between the first near point 321 and the second near point 322 is the smallest, such a second distance D2 being taken between these points orthogonally to the first sleeve end 31 and the second sleeve end 32.

Note that the ratio of first distance D1 to second distance D2 is preferentially between 8 and 16, and even more preferentially around 12 to within +/−10%.

Note also that the pair of distant points 311, 312 and the pair of near points 321, 322 are diametrically opposed with respect to the general extension axis A1.

It should also be noted that the pair of distant points 311, 312 is equidistant from the first tab 81 and the second tab 82.

It should also be noted that the pair of near points 321, 322 is equidistant from the first tab 81 and the second tab 82.

These arrangements are designed to optimize the positioning of the first hole 41 relative to the horse's eye, so that the shield 11, 12 forms a visual mask adapted to the visual field of the eye to which the shield 11, 12 is fitted.

It is also notable that each shield 11, 12 comprises at least two longitudinal bosses 9 which extend axially between the first sleeve end 31 and an outer collar edge 10. The longitudinal bosses 9 jointly border an inflection line 13 which also extends axially between the first sleeve end 31 and an outer collar edge 10. These arrangements are designed to give the shield 11, 12 a suitable grip on the horse's head 100.

The first hole 41 can be fitted with a corrective lens 410 to improve the vision of the horse's eye that the shield 11, 12 fitted with this corrective lens 410 is on.

In another embodiment, the shield 11,12 and the corrective lens 410 form a single-piece unit made from an optically corrective material. In this case, such an assembly is produced in a single molding pass, for example.

The shields 11, 12 are made by molding a plastic material.

These provisions are such that such a pair of goggles 1 is able to protect the horse's eyes from wind, dust, ultraviolet radiation, and excessive brightness when this is detected to be a source of discomfort and/or pain to the horse. In this way, said pair of goggles 1 can be used for a wide range of treatments in addition to those prescribed by an equine veterinary surgeon. Among the pathologies that the pair of goggles 1 can help to treat are corneal ulcers, keratitis and glaucoma, not to mention injuries that can be caused by a kick from another horse when the horses are in the pasture or being transported, or even frequent and pathological headshaking.

1. A pair of goggles (1) intended to be fitted to a head (100) of an equine, the pair of goggles (1) comprising a set of straps (20) and two shields (11, 12), each shield (11, 12) comprising a sleeve (3) which extends between a first sleeve end (31) and a second sleeve end (32), the first sleeve end (31) being provided with a first hole (41) and the second sleeve end (32) being provided with a second hole (42), characterized in that the second sleeve end (32) is provided with a collar (5) fitted to a rim (4) of the second hole (42).

2. The pair of goggles (1) according to claim 1, characterized in that the collar (5) and the sleeve (3) form a first angle (a) which lies between 90° and 120°.

3. The pair of goggles (1) according to claim 1, characterized in that the collar (5) extends between a topside (51) and an underside (52) which is equipped with a crown (6).

4. The pair of goggles (1) according to claim 3, characterized in that the rim (5) is provided with a plurality of channels (7) extending between the topside (51) and the underside (52) of the rim (5).

5. The pair of goggles (1) according to claim 3, characterized in that the channels (7) are radially interposed between the crown (6) and the rim (4) around a general extension axis (A1) of the sleeve (3).

6. The pair of goggles (1) according to claim 5, characterized in that the collar (5) comprises two tabs (81, 82) which are diametrically opposed to each other with respect to the general extension axis (A1).

7. The pair of goggles (1) according to claim 6, characterized in that a first tab (81) is arranged in a first plane (P1) and a second tab (82) is arranged in a second plane (P2), which form a second angle (B) between them, which is between 70° and 120°.

8. The pair of goggles (1) according to claim 1, characterized in that the sleeve (3) extends between at least one pair of distant points (311, 312) and one pair of near points (321, 322), the pair of distant points (311, 312) being whichever one of the respective pairs of points of the first sleeve end (31) and the second sleeve end (32) for which a first distance (D1) between a first distant point (311) and a second distant point (312) is greatest, the second pair of near points (321, 322) being whichever one of the respective pairs of points of the first sleeve end (31) and the second sleeve end (32) for which a second distance (D2) between a first near point (321) and a second near point (322) is smallest, a ratio of the first distance (D1) to the second distance (D2) is between 8 and 16.

9. The pair of goggles (1) according to claim 1, characterized in that each shield (11, 12) comprises at least two longitudinal bosses (9) which extend between the first sleeve end (31) and an outer collar edge (10) and which jointly border an inflection line (13) which also extends between the first sleeve end (31) and an outer collar edge (10).

10. The pair of goggles (1) according to claim 1, characterized in that the first hole (41) is provided with a corrective lens (410).
